(19) United States
(12) Patent Application Publication
(10) Pub. No.: US 2025/0368687 A1
(43) Pub. Date: Dec. 4, 2025

(54) SHORT SYNTHETIC PEPTIDES AND USES THEREOF IN THE TREATMENT OF ENTEROVIRUS INFECTION

(71) Applicant: CITY UNIVERSITY OF HONG KONG, Kowloon (HK)

(72) Inventors: Mingliang HE, Kowloon (HK); Mandi WU, Kowloon (HK)

(21) Appl. No.: 19/224,570

(22) Filed: May 30, 2025

Related U.S. Application Data
(60) Provisional application No. 63/654,554, filed on May 31, 2024.

(51) Int. Cl. C07K 14/00; A61K 38/00; A61K 45/06; A61P 31/14

(52) U.S. Cl. CPC C07K 14/001; A61K 45/06; A61P 31/14; A61K 38/00

(57) ABSTRACT

Disclosed herein are synthetic peptides and compositions comprising the same for the treatment of enterovirus infection. Also disclosed herein are methods of treating enterovirus infection by administering to a subject in need of such treatment a composition containing an effective amount of a synthetic peptide of the present disclosure. In some cases, the subject is a human.

### CROSS REFERENCE TO RELATED APPLICATIONS

This application relates to and claims the benefits of U.S. Provisional Application No. 63/654,554 filed May 31, 2024, the content of which is incorporated herein by reference in its entirety.

### REFERENCE TO A SEQUENCE LISTING

The present application is being filed along with a Sequence Listing in an electronic format. The Sequence Listing is provided as a file entitled “HP0355US_SeqList”, created May 4, 2025, which is 14 KB in size. The information in the electronic format of the Sequence Listing is incorporated herein by reference in its entirety.

### BACKGROUND OF THE INVENTION

### 1. Field of the Invention

The present disclosure in general relates to synthetic peptides having the antiviral activity, thus are useful in the field of the treatment or prophylaxis of viral infection.

### 2. Description of Related Art

Enterovirus A71 (EV-A71), a positive-sense, single-stranded RNA virus [(+) ssRNA virus) in the Picornaviridae family, is regarded as the main etiological pathogen responsible for human hand-foot-and-mouth disease (HFMD). HFMD is usually a self-limited disease accompanied by some mild symptoms, including fever, exanthema, and oral ulcers. It is also one of the most common neurotropic viruses to cause severe central nervous system (CNS) complications, including aseptic meningitis, acute flaccid paralysis, brainstem encephalitis, heart failure, and even death. EV-A71 infects millions of children and causes hundreds to thousands of deaths in China annually. In response to the outbreak, China has approved EV-A71 C4 genotype-based vaccines exclusively for children, making it the world's only licensed vaccine for controlling EV-A71. Supportive therapy remains the primary treatment approach for EV-A71 infection, because there is no approved therapeutic drug to combat EV-A71 infections.

Positive-sense ssRNA viruses (e.g., picornaviruses, flaviviruses, coronaviruses) rely on the cytosol heterogenous nuclear ribonucleoproteins (hnPNPs) to initiate translation polypeptide for viral replication. After entry and uncoating, EV-A71 initiates its life cycle through an internal ribosome entry site (IRES) directed viral protein translation. The IRES is located in the 5′-UTR of EV-A71, which cap-independently recruits 40S ribosomal components with the help of several host RNA binding proteins known as IRES trans-acting factors (ITAFs). These ITAFs bind to viral RNA across multiple domains and act to stabilize the structure of IRES when recruiting canonical translation factors and ribosomal subunits. A number of ITAFs have been identified to be involved in EV-A71 and other viruses' replication, such as hnRNP A1, FBP1, hnRNPK, AUF1, etc. hnRNP A1, a regulator of alternative splicing in the nucleus, is reported to directly bind with IRES as a noncanonical ITAF, changing the conformation for initiation of viral translation. As a (+) ssRNA virus, EV-A71 completes its life cycle in the cytosol, and the relocalization of hnRNP Al is of extreme importance for viral protein translation and genome RNA replication. It is reported that viral protease 2A^{pro} or 3C^{pro} cleaves the component proteins of the nuclear pore complex that may block the trafficking of hnRNP A1 between the nucleus and cytosol in the fast initiation of viral protein translation in this process. It has been reported that Hsp27 promotes EV-A71 propagation through enhancing viral IRES activity via 2A protease (2A^{pro})-mediated EIF4G cleavage and hnRNP A1 relocalization from the nucleus to the cytosol. However, the underlying mechanism is elusive.

Hsp27 works as a molecular chaperone in response to various stresses including pathogen infections. Hsp27 plays crucial roles in various types of cancers, inflammatory diseases, neurological diseases, the immune response, and virus infections, such as hepatitis B Virus, Porcine circovirus type 2 (PCV2), and Dengue virus (DENV) infections. Hsp27 can be modulated by phosphorylation at serine 15, 78, and 82 in response to a variety of signals, including heat shock, oxidative stress, or growth factors. The mitogen-activated protein kinases associated protein kinases (MAPKAP kinases 2, 3), downstream of MAP p38 protein kinase, are responsible for the phosphorylation of Hsp27. Various stimuli, including DNA damage, inflammatory cytokines, and viral infections, can trigger the activation of the p38 MAPK pathway. Multiple studies have shown that p38 MAPK signaling was activated by different types of viruses, including EV-A71, SARS-COV-2, ZIKV, and Junin Virus (JUNV). The p38 MAPK inhibitor, SB203580, reduced viral replication and inhibited the secretion of inflammatory factors (such as IL-6, IL-10, and TNF-α), the main cause of viral pathogenesis and death.

In the present disclosure, the inventors unexpectedly identify short synthetic peptides that may suppress Hsp27 phosphorylation and hnRNP A1 redistribution triggered by EV-A71 infection or ectopic 2A^{pro} expression and virus replication. Accordingly, these short synthetic peptides are candidate compounds for the development of a medicament for treating EV-A71infections.

### SUMMARY

In general, the present disclosure relates to the development of novel compounds and/or methods for treating an enterovirus (e.g., EV-A71) infection.

Accordingly, the first aspect of the present disclosure aims at providing a short synthetic peptide capable of treating an enterovirus (e.g., EV-A71) infection. The short synthetic peptide consists of the amino acid sequence set forth as PKKRRQRRRAYSRAX1X2X3QLX4S (SEQ ID NO: 1), wherein,
X₁ is leucine (L) or alanine (A);
X₂ and X₄ are independently serine(S) or alanine (A);
X₃ is arginine (R) or alanine (A); and
the N-terminus of the amino acid sequence is acetylated and the C-terminus of the amino acid sequence is amidated.

According to one preferred embodiment, X₁ is L, X₂ and X₄ are independently S, and X₃ is R, and the synthetic peptide consists of the amino acid sequence of SEQ ID NO: 2 (hereinafter “S78”).

According to another preferred embodiment, X₁ is L, X₂ is A, X₃ is R, and X₄ is S, and the synthetic peptide consists of the amino acid sequence of SEQ ID NO: 3 (hereinafter “S78A”).

According to a further preferred embodiment, X₁ is L, X₂ is S, X₃ is R, and X₄ is A, and the synthetic peptide consists of the amino acid sequence of SEQ ID NO: 4 (hereinafter “S82A”).

According to a further preferred embodiment, X₁, X₂, and X₃ are independently A, and X₄ is S, and the synthetic peptide consists of the amino acid sequence of SEQ ID NO: 5 (hereinafter “S78-3A”).

The second aspect of the present disclosure aims at providing a medicament and/or a composition suitable for treating an enterovirus infection. The medicament or composition comprises the synthetic peptide described above, and a pharmaceutically acceptable carrier.

According to one preferred embodiment, the synthetic peptide consists of the amino acid sequence of SEQ ID NO: 2 (hereinafter “S78”).

According to another preferred embodiment, the synthetic peptide consists of the amino acid sequence of SEQ ID NO: 3 (hereinafter “S78A”).

According to a further preferred embodiment, the synthetic peptide consists of the amino acid sequence of SEQ ID NO: 4 (hereinafter “S82”).

According to a further preferred embodiment, the synthetic peptide consists of the amino acid sequence of SEQ ID NO: 5 (hereinafter “S78-3A”).

The medicament or composition of the present disclosure may be administered to the subject via intravascular delivery (e.g., injection or infusion), oral, enteral, rectal, pulmonary (e.g., inhalation), nasal, topical (including transdermal, buccal and sublingual), intravesical, intravitreal, subconjunctival, intraperitoneal, vaginal, brain delivery (e.g., intracerebroventricular, and intracerebral), CNS delivery (e.g., intrathecal, peri-spinal, and intra-spinal) or parenteral (e.g., subcutaneous, intramuscular, intravenous, and intradermal), transmucosal administration or administration via an implant, or other delivery routes known in the art.

The third aspect of the present disclosure is thus directed to a method of treating a subject suffering from enterovirus infection (e.g., EV-A71 infection). The method comprises administering to the subject a medicament or a composition of the present disclosure described above for ameliorating or alleviating symptoms related to the enterovirus infection.

According to optional embodiments of the present disclosure, the method further comprises administering to the subject an antiviral agent before, together with, or after administering the medicament or the composition of the present disclosure described above.

Examples of the antiviral agent suitable for use in the present method include, but are not limited to, berberine, glycyrrhizic acid, lactoferrin, kaempferol, melittin, minocycline, quinacrine, quercetin, reservatrol, ribavirin, suramin, ursolic acid, vapendavir, and xiyanping.

In all embodiments, the subject is a human.

Many of the attendant features and advantages of the present disclosure will become better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1 Requirement of Hsp27 Phosphorylation for its nuclear localization upon EV-A71 infection. RD cells were treated with SB203580 at indicated concentration for 2 hours, then infected with EV-A71 at the MOI of 10 for 6 hours. Cell lysates were collected for western blot assay. Hsp27 phosphorylation on Ser 15, 78, 82 were detected by specific antibodies. GAPDH were detected as internal control.

FIGS. 2A-2B SB 203580 Inhibited EV-A71-induced Hsp27 phosphorylation at Ser 78 and colocalization of Hsp27 and DAPI. FIG. 2A Densitometric analysis of the phosphorylation level at Ser 78 in FIG. 1A was quantified and normalized with GAPDH using ImageJ. FIG. 2B Colocalization analysis of confocal images in FIG. 1B using the JACoP-plugin of the extended ImageJ version Fiji. The MI colocalization coefficient (the fraction of Hsp27 in DAPI) was computed. M1 of Hsp27 overlapping DAPI without EV-A71 infection showed no significance compared with control (0 μM). Statistical analyses were carried out using Student's t-test. *: p<0.05, **: p<0.01, ***: p<0.001. Data are expressed as mean ±SD.

FIGS. 3A-3B Ser78 phosphorylation of Hsp27 is critical for induction of hnRNP A1 cytosol redistribution upon EV-A71 Infection. (FIG. 3A) Colocalization analysis of FIGS. 1C, 2, and 3 was conducted using the JACoP-plugin of the extended ImageJ version Fiji. The M1 colocalization coefficient (the fraction of Hsp27 in Hoechst) was computed. (FIG. 3B) The fraction of hnRNP A1 in Hoechst was quantified in Hsp27 restoring cells with or without EV-A71 infection. Statistical analyses were carried out using Student's t-test. *: p<0.05, **: p<0.01. Data are expressed as mean ±SD.

FIGS. 4A-4B Phosphorylation-mimic of Hsp27 cannot induce hnRNP A1 Translocation without EV-A71 Infection. (FIG. 4A) Colocalization analysis was conducted using the JACoP-plugin of the extended ImageJ version Fiji. The M1 colocalization coefficient (the fraction of Hsp27 in Hoechst) was computed. (FIG. 4B) The fraction of hnRNP A1 in Hoechst was quantified in Hsp27-restoring cells with or without EV-A71 infection. Statistical analyses were carried out using Student's t-test. *: p<0.05, **: p<0.01, ***: p<0.001. Data are expressed as mean ±SD.

FIGS. 5A-5D Quantification of colocalization of Hsp27, hnRNP A1 and hoechst in Hsp27-restoring cells with ectopic expression of 2A^{Pro} and 2A^{C110A}. The JACoP-plugin of the extended ImageJ version Fiji was used to compute the M1 colocalization coefficient (the fraction of Hsp27 in Hoechst or the fraction of hnRNP A1 in Hoechst). FIG. 5A-5D. The fraction of Hsp27 in Hoechst or the fraction of hnRNP A1 in Hoechst was quantified in Hsp27-restoring cells transfected with 2A^{pro} or 2A^{C110A}. Statistical analyses were carried out using Student's t-test. *: p<0.05, **: p <0.01. Data are expressed as mean ±SD.

FIGS. 6A-6C Ser78 phosphorylation-deficient Hsp27 suppresses the viral IRES activity. (FIG. 6A) RD cells were pretreated with 2 μM SB203580 for 2 h, then co-transfected with a 2A^{pro}-expressing plasmid (200 ng) and pIRES reporter plasmid (200 ng) for 24 h, then the luciferase activity was measured. (FIG. 6B) HEK 293T cells were co-transfected with Hsp27 (WT, 3A, S15A, S78A, and S82A) plasmid (800 ng), pIRES reporter plasmid (200 ng) and 2A^{pro}-expressing plasmid (200 ng) for 24 h, then the luciferase activity was measured. (FIG. 6C) HEK 293T cells were co-transfected with Hsp27 (WT, 3A, S15A, S78A, 3D S15D, and S78D) plasmid (800 ng), pIRES reporter plasmid (200 ng) and 2A^{C110A}-expressing plasmid (200 ng) for 24 h, then the luciferase activity was measured. Statistical analyses were carried out using Student's t-test. *: p<0.05, **: p<0.01, ***: p<0.001. Data are expressed as mean ±SD.

FIGS. 7A-7D Ser78 phosphorylation-deficient Hsp27 inhibits EV-A71 replication and propagation (FIG. 7A) HEK 293T cells were transfected with 500 ng Hsp27 (WT, 3A, S15A, S78A, S82A) plasmids for 48 hours, and then infected with EV-A71 at an MOI of 10 for 9h. Intracellular viral RNA were extracted and quantified by qPCR. (FIG. 7B) HEK 293T cells were transfected with 500 ng Hsp27 (WT, 3D, S15D, S78D, S82D) plasmids for 48 hours, and then infected with EV-A71 at an MOI of 10 for 9h. Intracellular viral RNA were extracted and quantified by qPCR. (FIG. 7C) HEK 293T cells were transfected with 500 ng Hsp27 (WT, S78A, S78D) plasmids for 48 hours, and treated with 8 μM SB203580 2 hours before infection with EV-A71. (FIG. 7D) HEK 293T cells were transfected with 500 ng Hsp27 (WT, 3A, S15A, S78A, S82A) plasmids for 48 hours, and then infected with EV-A71 at an MOI of 10 for 12h. Viral titration was calculated by TCID₅₀. Statistical analyses were carried out using Student's t-test. *: p<0.05, **: p<0.01, ***: p<0.001. Data are expressed as mean ±SD.

FIGS. 8A-8B Peptide S78 suppressed EV-A71-induced Hsp27/hnRNP A1 relocation. (FIG. 8A) Colocalization analysis of FIG. 7C was conducted using the JACoP-plugin of the extended ImageJ version Fiji. The M1 colocalization coefficient (the fraction of Hsp27 in Hoechst) was computed. (FIG. 8B) The fraction of hnRNP A1 in Hoechst was quantified. Statistical analyses were carried out using Student's t-test. *: p<0.05, **: p<0.01, ***: p<0.001. Data are expressed as mean ±SD.

FIGS. 9A-9B Peptide S78 blocks the 2A^{pro}-induced relocalization of Hsp27 and hnRNP A1. RD cells on the coverslips were treated with peptides (S78, S78A, and S82A) at the concentration of 25 μM for 2 hours, and then transfected with 2A^{pro} for 24 hours. The cells were fixed and stained with anti-Hsp27and anti-hnRNPAI, followed by Alexa Fluor 488-conjugated anti-rabbit antibody and Alexa Fluor 594-conjugated anti-mouse antibody. The nuclei were stained with Hoechst. The images were captured by Nikon A1HD25 Confocal Microscope. The JACoP-plugin of the extended ImageJ version Fiji was used to compute the M1 colocalization coefficient, in which (FIG. 9A) is M1 colocalization coefficient for the fraction of Hsp27 in Hoechst and (FIG. 9B) is the M1 colocalization coefficient for the fraction of hnRNP A1 in Hoechst. Statistical analyses were carried out using Student's t-test. *: p<0.05, **: p<0.01, ***: p<0.001. Data are expressed as mean ±SD.

FIGS. 10A-10K. Peptide S78 inhibited EV-A71 replication and propagation. (FIG. 10A) RD cells were pre-treated with peptides S78 or for 2 hours and infected with EV-A71 at the MOI of 10 for 6 hours. Cell lysates were collected for western blot assay. Hsp27 phosphorylation on Ser 15, 78, 82 were detected by specific antibodies. β-Actin were detected as internal control. (FIG. 10B) Densitometric analysis of FIG. 9A. (FIG. 10C) HEK 293T cells were pre-treated with peptides S78 or S78A at the concentration of 25 μM for 2 hours and then co-transfected with Hsp27-WT plasmid (800 ng), pIRES reporter plasmid (200 ng) and 2A^{pro}-expressing plasmid or 2A_{C110A}-expressing plasmid (200 ng) for 24 h, then the luciferase activity was measured. (FIG. 10D-10E) RD cells were treated with peptides S78 or S78A and then infected with EV-A71. EV-A71 VP1 were detected. (FIG. 10F) RD cells were treated with peptides and then infected with EV-A71 at the MOI of 1 for 12 hours. The EV-A71-induced CPE was captured. (FIG. 10G-10H) Intracellular viral RNA was extracted and quantified by qPCR. (FIG. 10I) Densitometric analysis FIG. 9D-E. (FIG. 10J) RD cells were treated with peptides S78 or and then infected with EV-A71 at the MOI of 1 for 12 hours. Extracellular viral RNA was extracted and quantified by qPCR. (FIG. 10K) Viral titration was calculated by TCID₅₀. The Statistical analyses were carried out using Student's t-test. *: p<0.05, **: p<0.01, ***: p<0.001. Data are expressed as mean ±SD.

FIGS. 11A-11G Inhibition of EV-A71 Protein Translation and Viral Reproduction by peptide S82A. (FIG. 11A) HEK 293T cells were pre-treated with peptides NC, S78, S78A, S82A, or S78-3A at the concentration of 25 μM for 2 hours and then co-transfected with Hsp27-WT plasmid (800 ng), pIRES reporter plasmid (200 ng) and 2A^{pro}-expressing plasmid for 24 h, then the luciferase activity was measured. (FIG. 11B) RD cells were pre-treated with peptides NC at indicated concentrations for 2 hours and infected with EV-A71 at the MOI of 1 for 9 hours. Intracellular viral RNA was extracted and quantified by qPCR. (FIG. 11C-11E) RD cells were treated with peptides NC, S82A, or S78-3A at indicated concentrations for 2 hours and then infected with EV-A71 at the MOI of 1 for 9 hours. Cell lysates were collected for western blot assay. EV-A71 VP1 were detected. β-Actin was detected as an internal control. (FIG. 11F) Densitometric analysis of the EV-A71 VP1 level in FIG. S7C-E was quantified and normalized with β-Actin using ImageJ. (FIG. 11G) RD cells were treated with peptides S78, S78A, S82A, or S78-3A at the concentration of 25 μM for 2 hours and then infected with EV-A71 at the MOI of 0.1 for 24 hours. Viral titration was calculated. The Statistical analyses were carried out using Student's t-test. *: p<0.05, **: p<0.01, ***: p<0.001. Data are expressed as mean ±SD.

In accordance with common practice, the various described features/elements are not drawn to scale but instead are drawn to best illustrate specific features/elements relevant to the present disclosure.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

As used herein, the term “peptide” denotes a polymer of amino acid residues. By the term “synthetic peptide” as used herein, it is meant a peptide which does not comprise an entire naturally occurring protein molecule. The peptide is “synthetic” in that it may be produced by human intervention using such techniques as chemical synthesis, recombinant genetic techniques, or fragmentation of whole antigen or the like. Throughout the present disclosure, the positions of any specified amino acid residues within a peptide are numbered starting from the N terminus of the peptide. When amino acids are not designated as either D- or L-amino acids, the amino acid is either an L-amino acid or could be either a D-or L-amino acid, unless the context requires a particular isomer. Further, the notation used herein for the polypeptide amino acid residues are those abbreviations commonly used in the art.

As discussed herein, minor variations in the amino acid sequences of proteins/peptides are contemplated as being encompassed by the presently disclosed and claimed inventive concept(s), providing that the variations in the amino acid sequence maintain at least 90%, such as at least 70%, 71%, 72%, 73%, 75%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99%. The present synthetic peptide may be modified specifically to alter a feature of the peptide unrelated to its physiological activity. For example, certain amino acids can be changed and/or deleted without affecting the physiological activity of the peptide in this study (i.e., its ability to treat enteroviral infection). In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the peptide derivative. Fragments or analogs of proteins/peptides can be readily prepared by those of ordinary skill in the art. Preferred amino- and carboxy-termini of fragments or analogs occur near boundaries of functional domains.

The term “treatment” as used herein are intended to mean obtaining a desired pharmacological and/or physiologic effect, e.g., suppressing the replication and/or propagation of enterovirus. The effect may be prophylactic in terms of completely or partially preventing a disease or symptom thereof and/or therapeutic in terms of a partial or complete cure for a disease and/or adverse effect attributable to the disease. “Treatment” as used herein includes preventative (e.g., prophylactic), curative or palliative treatment of a disease in a mammal, particularly human; and includes: (1) preventative (e.g., prophylactic), curative or palliative treatment of a disease or condition (e.g., retinal degeneration or tissue injury) from occurring in an individual who may be pre-disposed to the disease but has not yet been diagnosed as having it; (2) inhibiting a disease (e.g., by arresting its development); or (3) relieving a disease (e.g., reducing symptoms associated with the disease).

The term “administered”, “administering” or “administration” are used interchangeably herein to refer a mode of delivery of an agent (e.g., a compound or a composition) of the present disclosure, including, without limitation, intravascular delivery (e.g., injection or infusion), oral, enteral, rectal, pulmonary (e.g., inhalation), nasal, topical (including transdermal, buccal and sublingual), intravesical, intravitreal, subconjunctival, intraperitoneal, vaginal, brain delivery (e.g., intracerebroventricular, and intracerebral), CNS delivery (e.g., intrathecal, peri-spinal, and intra-spinal), parenteral (e.g., subcutaneous, intramuscular, intravenous, and intradermal), transmucosal administration or administration via an implant, or other delivery routes known in the art.

The term “an effective amount” as used herein refers to an amount effective, at dosages, and for periods of time necessary, to achieve the desired result with respect to the treatment of a disease. For example, in the treatment of a retinal degenerative disease, an agent (i.e., a compound, a synthetic peptide, or a nucleic acid encoding a therapeutic peptide) which decreases, prevents, delays or suppresses or arrests any symptoms of the enteroviral infection would be effective. Similarly, in the treatment of a condition in need of suppressing replication and/or propagation of enterovirus, an agent (i.e., a compound, a synthetic peptide, or a nucleic acid encoding a therapeutic peptide) which decreases, prevents, delays or suppresses or arrests any symptoms of the condition or promotes the suppression of replication and/or propagation of enterovirus would be effective. An effective amount of an agent is not required to cure a disease or condition but will provide a treatment for a disease or condition such that the onset of the disease or condition is delayed, hindered or prevented, or the disease or condition symptoms are ameliorated. The effective amount may be divided into one, two or more doses in a suitable form to be administered at one, two or more times throughout a designated time.

The term “subject” or “patient” is used interchangeably herein and is intended to mean a mammal including the human species that is treatable by the synthetic peptide and/or method of the present disclosure. The term “mammal” refers to all members of the class Mammalia, including humans, primates, domestic and farm animals, such as rabbit, pig, sheep, and cattle; as well as zoo, sports or pet animals; and rodents, such as mouse and rat. Further, the term “subject” or “patient” intended to refer to both the male and female gender unless one gender is specifically indicated. Accordingly, the term “subject” or “patient” comprises any mammal which may benefit from the treatment method of the present disclosure. Examples of a “subject” or “patient” include, but are not limited to, a human, rat, mouse, guinea pig, monkey, pig, goat, cow, horse, dog, cat, bird and fowl. In an exemplary embodiment, the patient is a human.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The singular forms “a”, “and”, and “the” are used herein to include plural referents unless the context clearly dictates otherwise.

### II. The Present Synthetic Peptide

The present disclosure therefore aims at providing a short synthetic peptide capable of treating an enterovirus (e.g., EV-A71) infection. The short synthetic peptide consists of the amino acid sequence set forth as PKKRRQRRRAYSRAX1X2X3QLX4S (SEQ ID NO: 1), wherein,
X₁ is leucine (L) or alanine (A);
X₂ and X₄ are independently serine(S) or alanine (A); and
X₃ is arginine (R) or alanine (A).

Alternatively or optionally, the N-terminus of the amino acid sequence of the synthetic peptide is acetylated and the C-terminus of the amino acid sequence is amidated.

According to one preferred embodiment, the synthetic peptide of the present disclosure has the amino acid sequence of RKKRRQRRRAYSRALSRQLSS (SEQ ID NO: 2, “S78”).

According to another preferred embodiment, the synthetic peptide of the present disclosure has the amino acid sequence of RKKRRQRRRAYSRALARQLSS (SEQ ID NO: 3, “S78A”).

According to a further preferred embodiment, the synthetic peptide of the present disclosure has the amino acid sequence of RKKRRQRRRAYSRALSRQLAS (SEQ ID NO: 4, “S82A”).

According to a further preferred embodiment, the synthetic peptide of the present disclosure has the amino acid sequence of RKKRRQRRRAYSRAAAAQLSS (SEQ ID NO: 5, “S78-3A”).

The present synthetic peptide may be synthesized in accordance with any standard peptide synthesis protocol in the art. For example, the present synthetic peptides may be synthesized by using a solid-phase peptide synthesizer (ABI433A peptide synthesizer, Applied Biosystems Inc., Life Technologies Corp., Foster City, CA, USA) in accordance with the manufacturer's protocols.

Alternatively, the present synthetic peptides may be prepared using recombinant technology. For example, one can clone a nucleic acid encoding the present peptide in an expression vector, in which the nucleic acid is operably linked to a regulatory sequence suitable for expressing the present peptide in a host cell. One can then introduce the vector into a suitable host cell to express the peptide. The expressed recombinant polypeptide can be purified from the host cell by methods such as ammonium sulfate precipitation and fractionation column chromatography. A peptide thus prepared can be tested for its activity according to the method described in the examples below.

The above-mentioned nucleic acids or polynucleotide can be delivered using polymeric, biodegradable microparticle or microcapsule delivery devices known in the art. Another way to achieve the uptake of the nucleic acid in a host is using liposomes, prepared by standard methods. The polynucleotide can be incorporated alone into these delivery vehicles or co-incorporated with tissue-specific antibodies. Alternatively, one can prepare a molecular conjugate composed of a plasmid or other vector attached to poly-L-lysine by electrostatic or covalent forces. Alternatively, tissue specific targeting can be achieved using tissue-specific transcriptional regulatory elements that are known in the art. Delivery of “naked DNA” (i.e., without a delivery vehicle) to an intramuscular, intradermal, or subcutaneous site is another means to achieve in vivo expression.

The present synthetic peptide may be modified at its N-terminus or C-terminus. Examples of N-terminal modifications include, but are not limited to, N-glycated, N-alkylated, and N-acetylated amino acid. A terminal modification can include pegylation. An example of C-terminal modification is a C-terminal amidated amino acid. Alternatively, one or more peptide bond may be replaced by a non-peptidyl linkage, the individual amino acid moieties may be modified through treatment with agents capable of reacting with selected side chains or terminal residues.

Various functional groups may also be added at various points of the synthetic peptide that are susceptible to chemical modification. Functional groups may be added to the termini of the peptide. In some embodiments, the function groups improve the activity of the peptide with regard to one or more characteristics, such as improving the stability, efficacy, or selectivity of the synthetic peptide; improving the penetration of the synthetic peptide across cellular membranes and/or tissue barrier; improving tissue localization; reducing toxicity or clearance; and improving resistance to expulsion by cellular pump and the like. Non-limited examples of suitable functional groups are those that facilitate transport of a peptide attached thereto into a cell, for example, by reducing the hydrophilicity and increasing the lipophilicity of the peptide, these functional groups may optionally and preferably be cleaved in vivo, either by hydrolysis or enzymatically, inside the cell. Hydroxy protecting groups include esters, carbonates and carbamate protecting groups. Amine protecting groups include alkoxy and aryloxy carbonyl groups. Carboxylic acid protecting groups include aliphatic, benzylic and aryl esters.

A “peptidomimetic organic moiety” can optionally be substituted for amino acid residues in the present synthetic peptide both as conservative and as non-conservative substitutions. The peptidomimetic organic moieties optionally and preferably have steric, electronic or configuration properties similar to the replaced amino acid and such peptidomimetics are used to replace amino acids in the essential positions, and are considered conservative substitutions. Peptidomimetics may optionally be used to inhibit degradation of peptides by enzymatic or other degradative processes. The peptidomimetics can optionally and preferably be produced by organic synthetic techniques. Non-limiting examples of suitable petidomimetics include isosteres of amide bonds, 3-amino-2-propenidone-6-carboxylic acid, hydroxyl-1,2,3,4-tetrahydro-isoquinoline-3-carboxylate, 1,2,3,4-tetrahydro-isoquinoline-3-carboxylate, and histidine isoquinolone carboxylic acid.

Any part of the synthetic peptide may optionally be chemically modified, such as by the addition of functional groups. The modification may optionally be performed during the synthesis of the present peptide. Non-limiting exemplary types of the modification include carboxymethylation, acylation, phosphorylation, glycosylation or fatty acylation. Ether bonds can optionally be used to join the serine or threonine hydroxyl to the hydroxyl of a sugar. Amide bonds can optionally be used to join the glutamate or aspartate carboxy groups to an amino group of sugar. Acetal and ketal bonds can also optionally be formed between amino acids and carbon hydrates.

### III. Compositions for the Treatment of Enteroviral Infection

The present synthetic peptides are suitable for treating a subject suffering from enterovirus infection, particularly, EV-A71 infection. Accordingly, a further aspect of the present disclosure is to provide a medicament comprising the present synthetic peptide for treating EV-A71 infection.

In one embodiment, the medicament is for the treatment of enterovirus infection, particularly, EV-A71 infection.

The medicament is manufactured by mixing suitable amount of the present synthetic peptide with a pharmaceutically acceptable carrier, excipient or stabilizer into a composition. In particular embodiments, the synthetic peptide is selected from the group of peptides as described above, which include but are not limited to, S78, S78A, S82A and S78-3A, and a combination thereof.

The amount of the peptide present in the medicament or the composition will depend on the peptide used. The peptide typically will be present in the composition in the amount from about 0.001% to about 10% by weight, such as 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2., 2.3, 2.4, 2.5, 2.6, 2.7, 2.8, 2.9, 3.9, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and 10.0% by weight; in particular in an amount from about 0.01% to about 5% by weight, such as 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2., 2.3, 2.4, 2.5, 2.6, 2.7, 2.8, 2.9, 3.9, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% by weight.

Pharmaceutical acceptable carriers, excipients or stabilizers for use with the synthetic peptides are well known in the relevant art, and include but are not limited to non-toxic inert solid, semi-solid, or liquid filler, diluent, encapsulating agent or formulation auxiliary. Typical pharmaceutically acceptable carrier is water or physiological saline. Examples of pharmaceutically acceptable carriers include, but are not limited to, sugars such as lactose, glucose, and sucrose; starches such as corn starch; cellulose and its derivatives such as carboxymethyl cellulose, ethyl cellulose, and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols such as propylene glycol; esters such as ethyl oleate and ethyl laurate; agar; buffering agents such as magnesium hydroxide and aluminum hydroxide; alginic acid; as well as other agents such as non-toxic lubricants (e.g., lauryl sulfate and magnesium stearate), coloring agents, releasing agents, flavoring agents, preservatives and antioxidants. The composition may further comprise an anti-biotic or an anti-mycotic agent therein.

Suitable routes of administration of the medicament or the composition of the present disclosure are intravascular delivery (e.g., injection or infusion), oral, enteral, rectal, pulmonary (e.g., inhalation), nasal, topical (including transdermal, buccal and sublingual), intravesical, intravitreal, intraperitoneal, vaginal, brain delivery (e.g., intracerebroventricular, and intracerebral), CNS delivery (e.g., intrathccal, perispinal, and intra-spinal) or parenteral (e.g., subcutaneous, intramuscular, intravenous, and intradermal), transmucosal administration or administration via an implant, or other delivery routes known in the art.

Pharmaceutical composition suitable for oral administration may be formulated into discrete dosage units such as pills, tablets, lozenges or hard or soft capsules, or as a dispersible powder or granules, or as solutions or suspensions for example, aqueous or oily suspensions, emulsions, syrups, elixirs, or enteral formulas. The composition may be presented in uni-dose or multi-dose containers, such as sealed vials or ampoules, and may be stored in a lyophilized condition requiring the addition of sterile liquid carrier (e.g., water or saline) prior to use.

Pharmaceutical composition suitable for parental administration may be formulated into aqueous or non-aqueous sterile injection by mixing or dispersing the present synthetic peptide with a sterile solvent, such as water, Ringer's solution, saline, 1,3-butanediol, alcohol etc. Alternatively, fixed oil, fatty acid or synthetic mono-or diglycerides may be used as the solvent. The composition may be sterilized by filtering through a filter.

For topical or transdermal application, the pharmaceutical composition is generally formulated into ointments, pastes, creams, lotions, gels, patches or sprays. Ophthalmic formulations, ear drops, and eye drops are also contemplated within the scope of the invention. Depending on the type and severity of the disease, about 1 μg/kg to about 100 mg/kg (e.g., 0.01-50 mg/kg) of the present synthetic peptide is administered to the patient, such as 1, 5, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 900, 910, 920, 930, 940, 950, 960, 970, 980, 990, or 1,000 μg/kg; or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/kg. A typical daily or weekly dosage might range from about 0.01 mg/Kg to about 50 mg/kg or more, such as 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20,, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg/Kg. The doses utilized for any of the above-described purposes of topical administration will generally be administered one to several, e.g., four, six, eight or even more, times per day.

Pharmaceutical composition suitable for pulmonary administration is formulated as find dust or mists which may be generated by means of metered dose pressurized aerosols, nebulizers or insufflators.

The pharmaceutical composition provided by the invention preferably is presented in the form of a kit. In the present disclosure, a “kit” is understood as a product containing the synthetic peptide(s) provided by the present disclosure and/or the additional therapeutic compounds forming the packaged composition such that the transport, storage and simultaneous or successive administration thereof is allowed. Therefore, the kits of the invention can contain one or more sealed ampoules respectively contain the synthetic peptides of the invention, and which can be prepared in a single dose or as multiple doses. The kit can additionally contain a vehicle suitable for solubilizing the synthetic peptides such as aqueous media such as saline solution, Ringer's solution, dextrose and sodium chloride; water-soluble media such as alcohol, polyethylene glycol, propylethylene glycol; and water-insoluble vehicles if necessary. Another component which may be present in the kit is a package which allows maintaining the compositions of the invention within determined limits. Materials suitable for preparing such packages include glass, plastic (polyethylene, polypropylene, polycarbonate and the like), bottles, vials, paper, sachets and the like.

The kit of the invention can additionally contain instructions for the simultaneous, successive or separate administration of the different formulations present in the kit. Therefore, the kit of the invention can further comprise instructions for the simultaneous, successive or separate administration of the different components. Said instructions can be in the form of printed material or in the form of an electronic support which can store the instructions such that they can be read by a subject, such as electronic storage media (magnetic disks, tapes and the like), optical media (CD-ROM, DVD) and the like. The media can additionally or alternatively contain Internet webpages providing said instructions.

### IV. Methods for the Treatment of Enteroviral Infection

As it has been indicated above, the findings described in the present disclosure are useful for the prevention and/or treatment of enteroviral infection, such as infection caused by EV-A71.

The present disclosure therefore relates to a method for the prevention and/or treatment of EV-A71 infection, which comprises administering to a subject in need thereof a medicament or a composition described above, which comprises a synthetic peptide consisting of the amino acid sequence set forth as PKKRRQRRRAYSRAX1X2X3QLX4S (SEQ ID NO: 1), wherein,
X₁ is leucine (L) or alanine (A);
X₂ is serine(S) or alanine (A);
X₃ is arginine (R) or alanine (A); and
X₄ is serine(S) or alanine (A); and
a pharmaceutically acceptable carrier.

Alternatively or optionally, the N-terminus of the amino acid sequence of the synthetic peptide is acetylated and the C-terminus of the amino acid sequence is amidated.

The medicament and/or composition when administrated to the subject is capable of ameliorating or alleviating the symptoms associated with the retinal degenerative disease and/or the condition in need of tissue repair or regeneration.

In particular embodiments, the synthetic peptide is selected from the group of peptides described above, which include and are not limited to, S78, S78A, S82A S78-3A, and a combination thereof.

According to one embodiment, the present disclosure is related to a method for treating EV-A71 infection, which comprises administering to a subject in need thereof a medicament or a composition of the present disclosure.

Optionally or in addition, the method further includes the step of administering to the subject in need thereof an antiviral agent before, together with or after administering the medicament or the composition of the present disclosure.

Examples of the antiviral agent suitable for use in the present method include, but are not limited to, berberine, glycyrrhizic acid, lactoferrin, kaempferol, melittin, minocycline, quinacrine, quercetin, reservatrol, ribavirin, suramin, ursolic acid, vapendavir, and xiyanping.

In all embodiments, the subject suitable for treatment is a human.

The following Examples are provided to elucidate certain aspects of the present disclosure and to aid those of skilled in the art in practicing this invention. These Examples are in no way to be considered to limit the scope of the invention in any manner. Without further elaboration, it is believed that one skilled in the art can, based on the description herein, utilize the present disclosure to its fullest extent. All publications cited herein are hereby incorporated by reference in their entirety.

### EXAMPLE

### Materials and Methods

### Cells and Virus

HEK293T cells and RD cells (ATCC #CCL-136) were cultured in Dulbecco's modified Eagle's medium (DMEM) with 10% fetal bovine serum (FBS) and supplemented with 100U/mL of penicillin and 100 μg/mL of streptomycin. Hsp27 knockout RD cells (Hsp27-KO RD cells) were previously constructed using CRISPR/Cas 9 and maintained in the lab. The targeting single guide RNA (sgRNA) sequence was 5′-GCAUAGCCGCCUCUUCGACC-3′ (SEQ ID NO: 7). EV-A71 was obtained from Shenzhen Center for Disease Control and Prevention (SHZH98 strain, GenBank accession number AF302996) and propagated as previously described (Yi, Li. et al., Antivir Ther 2011 16(1): 51-58). The virus was aliquoted and stored at −80° C. until use.

### Plasmids

The mutants of Hsp27 (Hsp27-3A, S15A, S78A, S82A, 3D, S15D, S78D, and S82D) were constructed using a one-step mutagenesis kit (GeneTailor Site-Directed Mutagenesis System, Invitrogen, USA) on pcDNA4/HisMax B-Hsp27. The correction of construct was confirmed by automated DNA sequencing. The EV-A71 IRES reporter plasmid, pcDNA4/HisMax B-2A^{pro}, and pcDNA4/HisMax B-2A^{C110A} were constructed as previously described (Dong, Q. et al., Antiviral Res. 2018 150:39-46; Lu, J. et al., J. Virol. 2012 86(7):3767-3776). The protease-inactive mutation of 2A^{pro} (Cys¹¹⁰ to Ala¹¹⁰, 2A^{C110A}) was generated by site-directed mutagenesis with a one-step mutagenesis kit (Invitrogen). To construct EV-A71 IRES reporter plasmid, the Renilla Luciferase gene (RLuc) was inserted into pcDNA4/HisMax B between BamH I and EcoR V sites first, then the amplified IRES-FLuc encoding sequence was inserted downstream of the RLuc using EcoR V and Xbar I.

### Lentivirus Package

The GFP sequence of pLVTHM was substituted with the sequences encoding the wild type or mutants of Hsp27. Then, these plasmids were respectively transfected along with psPAX2 and pMD2.G lentivirus packaging system plasmids (psPAX2: pMD2.G: Plasmid of interest=6 μg: 3 μg: 9 μg) into HEK293T cells in 10-cm cell culture dishes using polyethylenimine (PEI). The medium was replaced after 2-4 hrs. After 48, 72, 96 hours, the supernatant of cells was collected, aliquoted, and stocked at −80° C.

### Immunofluorescence Assay

The cells on coverslips were infected with EV-A71 at the multiplicity of infection (MOI) of 40 for 6 hrs to assure the high efficiency of viral infection. Then the cells were fixed by 4% paraformaldehyde for 20 min, permeabilized by 0.5% Triton X-100 for 15 min, blocked with 5% BSA for 2 hrs, and stained by incubation with anti-Hsp27 (GTX101145) and anti-hnRNP A1 (sc-32301) antibody, then with Alexa Fluor 488 conjugated anti-rabbit antibody and Alexa Fluor 594 conjugated anti-mouse antibody respectively. After washing four times with 0.2% Triton X-100, the nuclei were stained with DAPI or Hoechst for 5 min. The images were captured by a Nikon A1HD25 High speed and Large Field of View Confocal Microscope. Colocalization analysis was conducted using the JACoP-plugin of the extended ImageJ version Fiji. The M1 colocalization coefficient was computed to illustrate the colocalization levels of Hsp27 and hnRNP A1 overlapped with Hoechst.

### Luciferase Assay

HEK 293T cells were seeded in 24-wells, and then co-transfected with plasmids to simultaneously express 2A^{pro} (200 ng), pIRES reporter (200 ng), and the wild-type Hsp27 or its mutants (800 ng). After culturing for 24 hours, the cell lysates were collected using a passive lysis buffer (Promega, USA). The Renilla luciferase (RLuc) and Firefly luciferase (FLuc) activity were determined by using a dual-luciferase reporter assay (Promega) according to the manufacturer's instructions in a Lumat LB9507 bioluminometer.

### Western Blot Assay

After cell lysis in Nonidet-P40 (NP-40) buffer, the cell lysates were centrifuged, and the concentration was determined by Bradford assay (Bio-Rad). 20 μg of protein was used for 12% SDS-PAGE and transferred onto polyvinylidene fluoride membrane (PVDF) (GE, MA, USA). Following 5% bovine serum albumin (BSA) blocking for 1 hrs, the membrane was incubated with antibodies (1:1000) against Hsp27 (sc-13132), pHsp27-Ser15 (ab76313), pHsp27-Ser78 (ADI-SPA-523), pHsp27-Ser82 (ab155987), VP1 (GTX132339), β-Actin (sc-47778), hnRNP A1 (sc-32301), or GAPDH (sc-32233) overnight in 4° C. After secondary antibodies (HRP-conjugated Affinipure Goat Anti-Mouse IgG(H+L), SA00001-1, and HRP-conjugated Affinipure Goat Anti-Rabbit IgG(H+L), SA00001-2) incubation for 1 hrs, the protein was visualized with a chemiluminescence reagent (PerkinElmer, MA, USA) on Bio-Rad Chemidoc Imaging System. The protein bands were quantified by densitometry with ImageJ if necessary.

### RNA Isolation

The intracellular RNA was isolated with TRIzol reagent (Ambion, Life, Technologies), and then 1 μg total RNA was used to synthesize cDNA using PrimeScript™ RT Master Mix (Takara) according to the manufacturer's instructions.

### Quantitative RT-PCR

Quantitative Real-time PCR (RT-qPCR) was performed using TB Green® Premix Ex Taq™ (Takara) on Applied Biosystems QuantStudio™M 3 Real-Time PCR Systems. The target fragment amplification was carried out as follows: initial activation at 95° C. for 30 s; PCR for 45 cycles: 95° C. for 5 s, 60° C. for 30 s. At the end of the amplification cycles, melting temperature analysis was carried out by a slow increase in temperature (0.1° C./s) up to 95° C. Primers of the target gene are listed in Table 1. The messenger RNA (mRNA) level of each target gene was normalized to the mRNA copies of GAPDH in the same sample.

TABLE 1
Primer sequences of target genes

| Name | Sequence (5′→3′) | | SEQ ID NO. |
|---|---|---|---|
| EV-A71 VP1 | forward primer | CGCATTGGGCGAGGTATC | 8 |
| | reverse primer | CGGACTGTAGGCACCTCGAA | 9 |
| GAPDH | forward primer | GTCTCCTCTGACTTCAACAGCG | 10 |
| | reverse primer | ACCACCCTGTTGCTGTAGCCAA | 11 |

### Virus Titration

RD cells were seeded into 96-well plates for 24 h, then cells were infected by 100 μL per well of serial 10-fold diluted supernatant in quintuplicate. The 50% tissue culture-infected dose (TCID₅₀) was calculated by the Kärber method after 120 h of infection.

### Peptide Synthesis and Treatment

The peptides were constructed and synthesized by GL Biochem (Shanghai) Ltd, and each peptide was modified by acetylation at the NH₂-termini and amidation at the COOH-termini for stability, and characterized by mass spectrometry (>95% purity).

To peptide treatment, RD cells were seeded into 24-well plates for 24 h, then cells were pre-treated with designated peptides at indicated concentrations for 2 hours and infected by EV-A71 or transfected by plasmids for following experiments. The amino acids sequences of the peptides are as listed in Table 2.

TABLE 2
The amino acids sequences of the peptides

| Peptide Name | Sequence | SEQ ID NO. |
|---|---|---|
| S78 | RKKRRQRRRAYSRALSRQLSS | 2 |
| S78A | RKKRRQRRRAYSRALARQLSS | 3 |
| S82A | RKKRRQRRRAYSRALSRQLAS | 4 |
| S78-3A | RKKRRQRRRAYSRAAAAQLSS | 5 |
| NC | RKKRRQRRRYSREANQPKPSPKRESGEE | 6 |

### CCK-8 Assay

RD cells were seeded in 96-well cell plates (1×10⁴/well) and placed in the 5% CO₂ incubator for 1 day. Then the cells were treated with peptides for 2 days. 90 μl medium and 10 μl of CCK-8 (BioSharp, BS350B) was added to each well of the 96-well plate, which were incubated at 37° C. for 1 h. A microplate reader (BioTek, Synergy H1) was used to measure the OD value of each well at 450 nm.

### Statistical Analysis

The results were expressed as mean±standard deviation (SD). All statistical analysis was carried out with GraphPad 8.0 software (GraphPad Inc.). Two-tailed Student's t-test was applied for two-group comparison. A p value<0.05 was considered statistically significant.

### Example 1: Phosphorylation was Required for Hsp27 Nuclear Localization Upon EV-A71 Infection

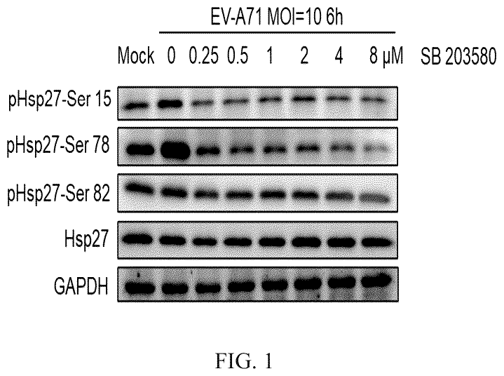

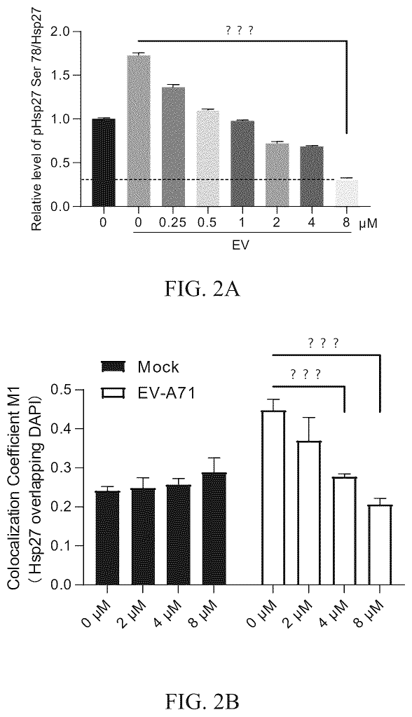

In this example, the effect of EV-A71 infection on Hsp27 phosphorylation was investigated. We first confirmed that EV-A71 infection increased the phosphorylation level of Hsp27 on serine 15 (Ser15), Ser78, and Ser82 (data not shown). Then, the phosphorylation status of Hsp27 upon virus infection under the treatment with a p38 inhibitor was examined, as the phosphorylation of Hsp27 could be resulted from p38 signaling activation. To this purpose, the cells were first treated with p38 kinase inhibitor −SB203580 at different concentrations (0, 0.25, 0.5, 1, 2, 4, and 8 μM) and the phosphorylation level of Hsp27 upon EV-A71 infection was examined by western blot assay. Results are predicted in FIGS. 1 and 2.

It was found that SB203580 inhibited the phosphorylation of Hsp27 at different degrees. The phosphorylation level slightly decreased at Ser15 and Ser82. Surprisingly, the phosphorylation at Ser78 was highly sensitive to SB203580 and markedly suppressed in a dose-dependent manner (FIG. 1). After quantification, with 8 μM of SB203580 treatment, the phosphorylation level at Ser78 reduced by over 85% (FIG. 2A). Hsp27 is predominantly located in the cytoplasm, however, stress (e.g., heat shock, virus infection) can induce partial redistribution of Hsp27 from the cytoplasm to the nucleus, concomitantly with its phosphorylation. We thus postulated that the cellular redistribution of Hsp27 may be associated with its phosphorylation upon EV-A71 infection. To verify the hypothesis, RD cells were with SB203580 at 0, 2, 4, and 8 μM for 2 h, and the cells were infected with EV-A71 for 6 h. After immunostaining, it was found that without EV-A71 infection, Hsp27 was not translocated to the nucleus, regardless of the treatment of cells with SB253080 (data not shown). Upon EV-A71 infection, a large amount of Hsp27 was translocated from cytosol to nucleus even without SB253080 treatment (data not shown), while a markedly reduced nuclear Hsp27 level was observed when the cells were treated with 2 μM of SB253080. When the concentration of SB253080 increased to 4 μM, the nuclear relocation of Hsp27 was further blocked to an invisible level (data not shown). The colocalization of Hsp27 and DAPI, which stands for the level of redistribution of Hsp27, was also quantified. The colocalization coefficient M1 was computed as the fraction of Hsp27 colocalized with DAPI in the total Hsp27 area using the JACoP-plugin in FIJI. Without SB203580 treatment, the fraction of Hsp27overlapping DAPI was 45%, while it was reduced in a dose-dependent manner. At 8 μM SB203580 treatment, the fraction of Hsp27 overlapping DAPI was even reduced to the basal level (FIG. 2B).

To further confirm the role of Hsp27 phosphorylation in its nuclear translocation, we constructed lentivirus vectors to restore the wild-type (WT) Hsp27 and phosphorylation-deficient mutant (Hsp27^{S15/78/82A}, Hsp27-3A) in Hsp27 knockout RD cells (Hsp27-KO RD cells) for three days. After EV-A71 infection at the MOI (multiplicity of infection) 40 for 6 h, the location of Hsp27 was examined using an immunofluorescence assay. The restored Hsp27 relocated from the cytoplasm into the nucleus 6 h post-infection (p. i.). However, little nuclear Hsp27-3A was observed, indicating the importance of Hsp27 phosphorylation on its nuclear translocation. (data not shown).

### Example 2: Ser78 Phosphorylation Played a Crucial Role in Hsp27 Nuclear Localization Upon EV-A71 Infection

To figure out the key phosphorylation site contributing to Hsp27 nuclear localization, phosphorylation-deficient Hsp27 mutants (Hsp27^{S15A}, Hsp27^{S78A}, and Hsp27^{S82A}) were restored in Hsp27-KO RD cells with lentivirus vectors for three days. The localization of the Hsp27 mutants was captured after EV-A71 infection for 6 h. It was found that both Hsp27^{S15A} and Hsp27^{S82A} relocated from the cytoplasm into the nucleus, while little Hsp27^{S78A} displayed in the nucleus (data not shown). These results indicated that the Ser78 phosphorylation is critical for Hsp27 nuclear localization upon EV-A71 infection.

### Example 3: Ser78 Phosphorylation of Hsp27 was Critical for Induction of hnRNP A1 Cytosol Redistribution Upon EV-A71 Infection

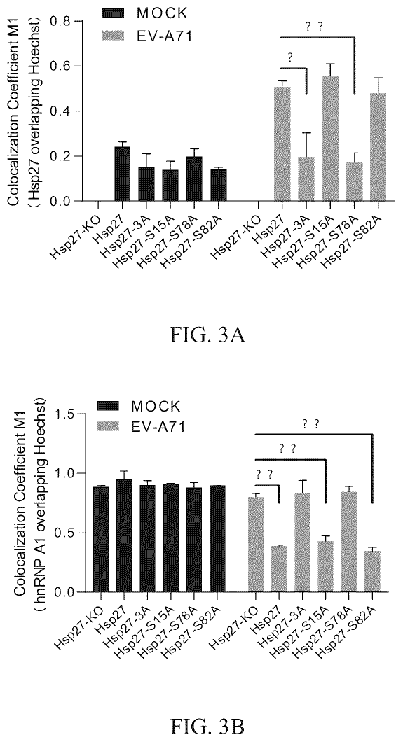

To dissect the effects of Hsp27 phosphorylation on hnRNP A1 cytosol redistribution, the wild type Hsp27 or a phosphorylation-deficient Hsp27 mutant (Hsp27-3A, Hsp27^{S15A}, Hsp27^{S78A}, and Hsp27^{S82A}) were restored in Hsp27-KO RD cells for 3 days, which were then infected with EV-A71 for 6 h. It was found that the cytosol redistribution of hnRNP A1 was completely blocked in the cells with the restoration of Hsp27^{S78A} expression but successfully rescued in the cytosol after the restoration of Hsp27^{S15A} and Hsp27^{S82A} expression. Results from quantification of colocalization showed that both Hsp27 and the phosphorylation-deficient Hsp27 mutants were mostly localized in the cytosol but not in the nucleus without EV-A71 infection; meanwhile, hnRNP A1 localized in the nucleus without cytosol distribution. Upon EV-A71 infection, a certain portion of Hsp27, Hsp27^{S15A}, and Hsp27^{S82A} translocated into the nucleus, and accordingly, hnRNP A1 was obviously redistributed in the cytosol. However, in Hsp27-3A or Hsp27^{S78A}-restored cells, the level of colocalization between Hsp27 and Hoechst failed to be increased by EV-A71 infection (FIG. 3A), and most hnRNP A1 was still colocalized with Hoechst (FIG. 3B). Taken together, the Ser78 phosphorylation of Hsp27 displayed a key role in both Hsp27 nuclear translocation and hnRNP A1 cytosol redistribution upon EV-A71 infection.

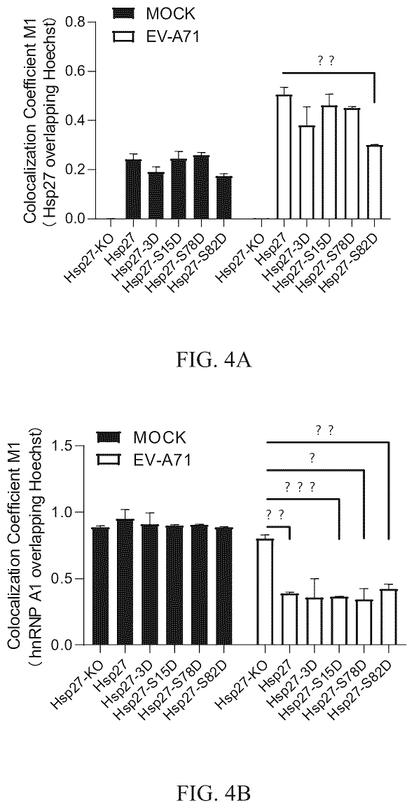

Further, whether the phosphorylated Hsp27 itself could direct its redistribution into the nucleus and induce hnRNP A1 cytosol redistribution without EV-A71 infection was investigated. Results are depicted in FIG. 4. It was found that Hsp27 or phosphorylation-active mimics (Hsp27-3D, Hsp27^{S15D}, Hsp27^{S755118D}, and Hsp27^{S82D}) did not translocate from the cytosol to the nucleus; meanwhile, they were not able to induce hnRNP A1 redistribution from the nucleus to cytosol without EV-A71 infection (data not shown). On the other hand, the phosphorylation-active Hsp27 mimics were mainly located in the nucleus upon EV-A71 infection; and obviously, hnRNP A1 redistributed in the cytosol upon EV-A71 infection as confirmed by the quantification analysis (FIG. 4A and 4B). Thus, phosphorylation of Hsp27 cannot directly drive the translocation of hnRNP A1 from the nucleus to cytosol without EV-A71 infection.

### Example 4: 2A^{pro} Protease Activity was Essential for Hsp27-Ser78 Phosphorylation-Mediated hnRNP A1 Cytosol Redistribution

It has been reported that Hsp27 promotes EV-A71 infections through 2A^{pro}-enhanced viral IRES activity. To further dissect the relationships among 2A^{pro}, Hsp27 phosphorylation, and hnRNP A1 cytosol redistribution, we restored Hsp27 and its phosphorylation-deficient mutants and then transfected a plasmid to express 2A^{pro} in Hsp27-KO cells.

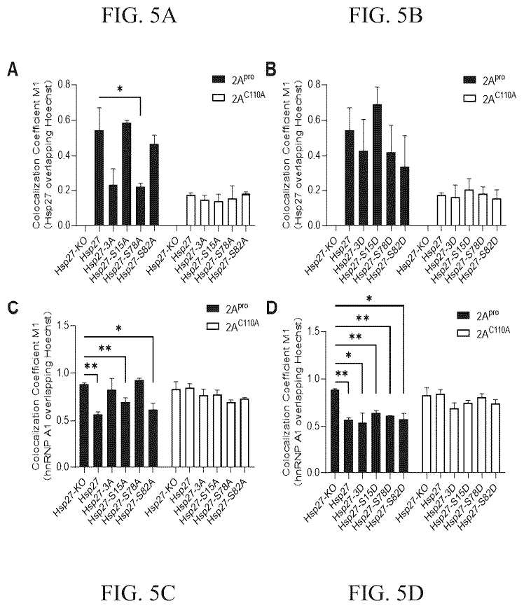

It was found that hnRNP A1 was fully localized in the nucleus in the control cells. 2A^{pro} successfully restored the cytosol redistribution of hnRNP A1 in the cells with Hsp27^{S15A} and Hsp27^{S82A} expression but failed to do so in the cells with Hsp27^{S78A} and Hsp27-3A expression (FIGS. 5A and 5C). Accordingly, 2A^{pro} obviously directed the cytosol redistribution of hnRNP A1 by phosphorylation-active Hsp27 mimics (Hsp27^{S15D}, Hsp27^{S78D}, Hsp27^{S82D}, and Hsp27-3D) in the Hsp27-KO RD cells (FIGS. 5 B and 5D).

To further examine the effect of protease activity on Hsp27 nuclear translocation and hnRNP A1 cytosol redistribution, the protease-deficient 2A^{C110A} mutant was co-expressed with cither wild-type Hsp27, phosphorylation-deficient Hsp27 mutants or phosphorylation-active Hsp27 mimics in the Hsp27-KO RD cells. Interestingly, 2A^{C110A} not only failed to induce Hsp27 translocation from the cytosol to the nucleus but also was not able to direct the redistribution of hnRNP A1 from the nucleus to the cytoplasm (FIG. 5), demonstrating an essential role of protease activity in 2A^{pro} for regulation of hnRNP A1 translocation.

### Example 5: Ser78 Phosphorylation Hsp27 was Crucial for Enhancing Viral IRES Activity

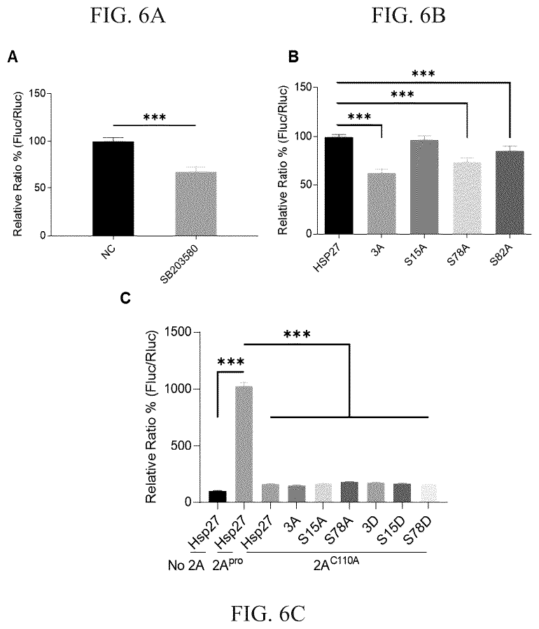

The effect of Ser78 phosphorylation on viral IRES activity was investigated in this example. To this purpose, RD cells were treated with SB203580 and co-transfected 2A^{pro}-expressing and an IRES-driven dicistronic reporter plasmids for 24 h. The renilla luciferase (RLuc) and firefly luciferase (FLuc) activities were determined with a dual-luciferase assay kit. The normalized ratio of Fluc to Rluc presented the relative IRES activity. Results are illustrated in FIG. 6.

It was found that after treating RD cells with 2 μM of SB203580, the IRES activity was reduced by 32% (FIG. 6A). Since HEK293T cells express little endogenous Hsp27, we thus examined the effects of phosphorylation-deficient Hsp27 on IRES activity in HEK293T cells. Phosphorylation-deficient Hsp27 expressing plasmids were co-transfected with 2A^{pro}-expressing and pIRES reporter plasmids into HEK293T cells for 24 h. Similar to SB203580, Hsp27-3A decreased the IRES activity by 37%. Strikingly, Ser78 phosphorylation-deficient Hsp27 (Hsp27^{S78A}) alone reduced the IRES activity by 25%, while Hsp27^{S82A} only marginally reduced the IRES activity (FIG. 6B). Surprisingly, Hsp27^{S15A} displayed no effects on the IRES activity. We thus examined the IRES activity of Hsp27-restoring cells with or without expression of 2A^{pro} or the inactive 2A^{pro} (2A^{C110A}). It was found that 2A^{pro} increased IRES activity by 10 folds, but 2A^{C110A} displayed almost no effects on the IRES activity in the cells expressed with the wild type Hsp27. Importantly, 2A^{C110A} had no effects on the IRES activity in the cells expressing phosphorylation-deficient or phosphorylation-mimic Hsp27 mutants (FIG. 6C), again demonstrating that phosphorylated Hsp27 cannot induce IRES-dependent protein translation without the help of 2A^{pro} or EV-A71.

Taken together, the Ser78-phosphorylation of Hsp27 was a key event to direct the hnRNP A1 cytosol relocalization and facilitate IRES-dependent translation upon EV-A71 infection.

### Example 6: Ser78 Phosphorylation-Deficient Hsp27 Inhibited EV-A71 Replication and Propagation

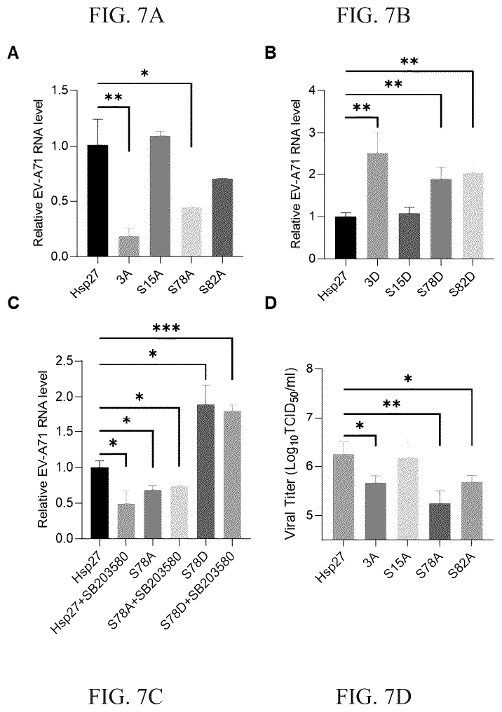

To explore the effects of Hsp27 phosphorylation on EV-A71 replication, Hsp27 and its phosphorylation-deficient mutants were ectopically expressed in 293T cells for 48 h, which were then infected with EV-A71 at the MOI of 1 for 9 h. The viral RNA level was measured by RT-qPCR. Compared with the control group in which Hsp27 was over-expressed, the viral RNA level decreased by 80% and 50% in cells ectopically expressed Hsp27-3A and Hsp27^{S78A} mutants, respectively (FIG. 7A). We noticed that the viral RNA level only slightly reduced in the Hsp27^{S82A}-expressing cells, whereas no difference of viral RNA levels was observed between the control and Hsp27^{S15A}-expressing groups (FIG. 7A). Accordingly, the viral RNA levels significantly increased about 150%, 90% and 100% in the groups with ectopic expression of phosphorylation-active Hsp27 mimics Hsp27-3D, Hsp27^{S78D}, and Hsp27^{S82D} respectively (FIG. 7B). Further, the effects of SB203580 on viral replication in the cells ectopic expressing Hsp27 were investigated. As shown in FIG. 7C, SB203580 decreased the viral RNA level by 50% in the Hsp27 over-expressing cells, but no obvious effects were obtained in the cells with Hsp27^{S78A}-and Hsp27^{S78D}-expressing cells. Again, these results demonstrated the crucial role of Ser78 phosphorylation in viral RNA replication, consistence with its nuclear localization and induction of hnRNP A1 cytosol redistribution.

To examine the effects on viral propagation, we over-expressed Hsp27 and phosphorylation-deficient Hsp27 mutants (Hsp27-3A, Hsp27^{S15A}, Hsp27^{S78A}, and Hsp27^{S82A}) in HEK293T cells, and then infected with EV-A71. The results showed that the Hsp27-3A and Hsp27^{S82A} reduced the viral titer by 75%, while the viral titer was not reduced by Hsp27^{S15A}. Strikingly, Hsp27^{S78A} reduced the viral titer by over 90% (FIG. 7D).

### Example 7: Peptide S78 Suppressed EV-A71 Induced Hsp27/hnRNP A1 Relocalization and Viral IRES Activity

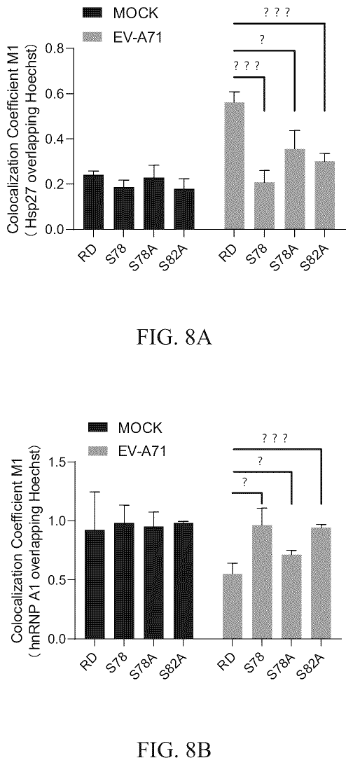

In this example, to demonstrate the antiviral potential by targeting Ser78 phosphorylation, three peptides (i.e., S78, S78A and S82A) tagged with TAT sequence for membrane penetration were designed and synthesized. We hypothesized that peptides S78 and S82A could interfere with Ser78 phosphorylation while peptides S78A (Ser78 replaced by Ala78) would not affect the phosphorylation of Ser78 of Hsp27. The CC₅₀ of these peptides was over 600 μM (data not shown), indicating no toxicity of these peptides in the test cells. RD cells were pre-treated with peptide S78, S78A or S82A at the concentrations of 25 μM for 2h and then infected with or without EV-A71 at an MOI of 40 for 6h. The localization of Hsp27 and hnRNP A1 in mock RD cells was not affected by the peptide treatment (data not shown). In the EV-A71-infected RD cells, the redistribution of both Hsp27 and hnRNP A1 were dramatically blocked by S78 and S82A treatment but only partially reduced by S78A (FIGS. 8A and 8B).

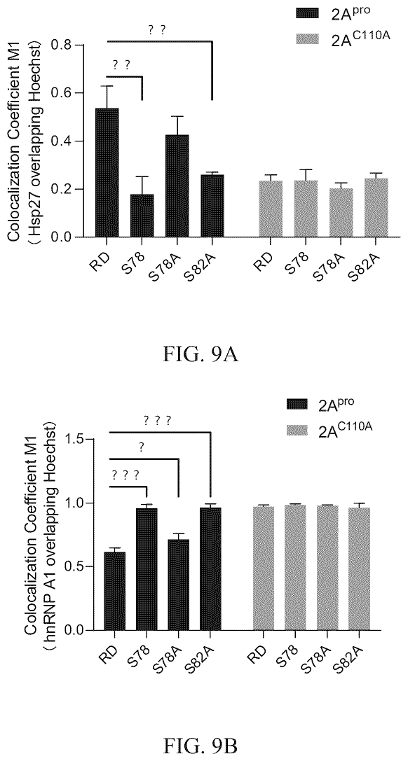

The effects of 2A^{pro} on the induction of Hsp27 and hnRNP A1 cellular redistribution were also investigated. In the peptide S78 or S82A-treated RD cells, the 2A^{pro}-induced relocalization of Hsp27 and hnRNP A1 was almost completely blocked, while S78A-treated cells still showed a certain cellular redistribution of both Hsp27 and hnRNP A1 (FIG. 9A and 9B). These results indicated that Ser82 played a less important role in 2A^{pro}-induced Hsp27 and hnRNP A1 relocalization. Consistent with finding in FIG. 5, 2A^{C110A} did not induce the re-localization of Hsp27, and hnRNP A1 was also not affected by any peptide treatment (data not shown). Two control peptides (NC, and S78-3A) were used to exclude potential nonspecific effects caused by peptide treatment. These peptides were also not toxic because they showed high CC₅₀ (data not shown). NC treatment did not affect EV-A71-induced Hsp27 and hnRNP A1 translocation, but S78-3A partially suppressed their redistribution as shown in the quantitative assay (data not shown).

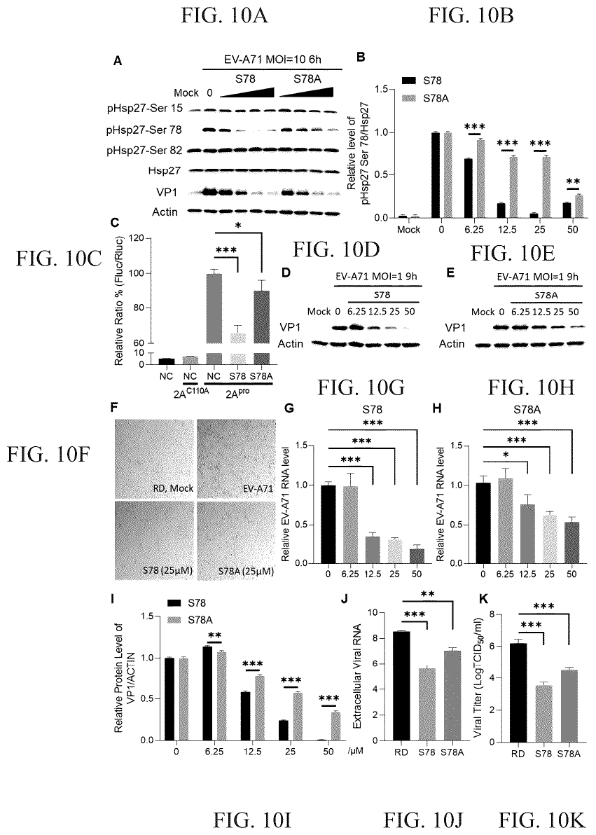

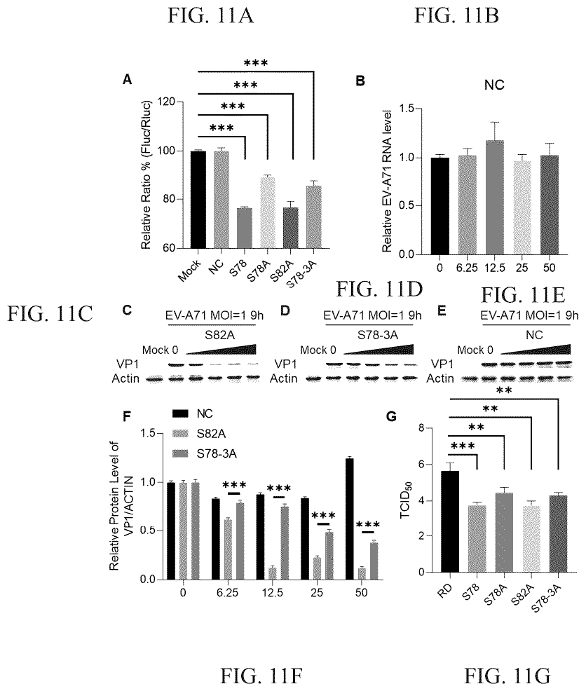

The phosphorylated level at Ser15, 78, or 82 of Hsp27 was detected by specific antibodies in the EV-A71 infected cells. S78 treatment dramatically inhibited Ser78phosphorylation rather than Ser15 or Ser82, while S78A peptide showed less inhibition on Ser78 phosphorylation (FIG. 10A and 10B). Furthermore, reporter assay showed that 2A^{pro}-induced viral IRES activity was decreased 34% in the S78-treated cells, similar to the level of SB203580 treatment (FIG. 5A). However, S78A treatment only slightly reduced about 10% of the IRES activity (FIG. 10C). As a negative control, NC did not affect viral IRES activity. The reduction in viral IRES activity by S82A treatment was similar to S78, while S78-3A mildly decreased the IRES activity (FIG. 11A), indicating the critical role of Ser78 phosphorylation for facilitating viral IRES-dependent protein translation.

### Example 8: Peptide S78 Inhibits EV-A71 Replication and Propagation

To further explore the potential antiviral effects of peptides S78 on EV-A71 infection, cytopathic effects (CPE) were examined on RD cells infected with EV-A71. S78 treatment dramatically protected the infected cells from EV-A71 infection, similar to the uninfected healthy RD cells, while S78A treatment showed mild protection from EV-A71-induced CPE (FIG. 10F). Moreover, the EV-A71 VP1 protein level was dramatically decreased by S78 in a dose-dependent manner. VP1 was even eliminated to the almost undetectable level at the concentration of 50 μM (FIG. 10D). However, the reduction of VP1 level was much weaker with S78A and S78-3A treatment (FIG. 10E, FIG. 11D). Compared to S78, S82A treatment showed a little bit weaker inhibition on VP1 expression (FIG. 11C). NC treatment had no effect on VP1 level (FIG. 11E). Densitometric assay showed that S78 and S82A reduced VP1 level by over 99% and 90% respectively, while S78A and S78-3A only decreased by about 60% at the concentration of 50 μM (FIG. 10I, FIG. 11F). S78 also dramatically reduced the viral RNA in a dose-dependent manner. At the concentration of 50 μM, the viral RNA was decreased by 82% (FIG. 10G), while S78A only reduced the viral RNA by 46% (FIG. 10H), while the NC peptide treatment had no effect on the viral RNA level (FIG. 11B). The S78 treatment decreased the extracellular viral RNA level by 800 folds at 25 μM, but S78A only reduced by 30 folds (FIG. 10J). The viral titer decreased 450 folds by S78 treatment, but only 50 folds by S78A treatment (FIG. 10K). S82A treatment had a similar effect on viral titer as compared to S78, while S78-3A only mildly decreased the viral titration (FIG. 11G). Taken together, the results further demonstrated that Ser78 phosphorylation plays a crucial role in facilitating EV-A71 infections and targeting Ser78 phosphorylation by S78 and S82A peptides displayed much stronger antiviral effects as compared with S78A peptide. S78 peptide could be a potential candidate for anti-EV-A71 drug development.

Taken together, results from the working examples demonstrated the present synthetic peptides derived from Hsp27 (e.g., S78, S78A, and S78-3A) could inhibit the replication and propagation of EV-A71 in its host cell via suppressing the phosphorylation of serine residue (position 78) of Hsp27 thereby prevents the nuclear translocation of Hsp27, introduction of hnRNP A1 cytosol relocation and IRES activity upon EV-A71 infection. Thus, the present synthetic peptides are potential candidates for the development of a medicament for the treatment of EV-A71 infection.

It will be understood that the above description of embodiments is given by way of example only and that various modifications may be made by those with ordinary skill in the art. The above specification provides a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A synthetic peptide consisting of the amino acid sequence set forth as PKKRRQRRRAYSRAX₁X₂X₃QLX₄S (SEQ ID NO: 1), wherein,
X₁ is leucine (L) or alanine (A);
X₂ and X₄ are independently serine(S) or alanine (A);
X₃ is arginine (R) or alanine (A); and
the N-terminus of the amino acid sequence is acetylated and the C-terminus of the amino acid sequence is amidated.

2. The synthetic peptide of claim 1, wherein X₁ is L, X₂ and X₄ are independently S, and X₃ is R, and the synthetic peptide consists of the amino acid sequence of SEQ ID NO: 2.

3. The synthetic peptide of claim 1, wherein X₁ is L, X₂ is A, X₃ is R, and X₄ is S, and the synthetic peptide consists of the amino acid sequence of SEQ ID NO: 3.

4. The synthetic peptide of claim 1, wherein X₁ is L, X₂ is S, X₃ is R, and X₄ is A, and the synthetic peptide consists of the amino acid sequence of SEQ ID NO: 4.

5. The synthetic peptide of claim 1, wherein X₁, X₂ and X₃ are independently A, and X₄ is S, and the synthetic peptide consists of the amino acid sequence of SEQ ID NO: 5.

6. A method of treating a subject having an enterovirus A71 (EV-A71) infection comprising administering to the subject an effective amount of the synthetic peptide of claim 1 to ameliorate or alleviate symptoms associated with the EV-A71 infection.

7. The method of claim 6, wherein the synthetic peptide consists of the amino acid sequence of SEQ ID NO: 2.

8. The method of claim 6, wherein the synthetic peptide consists of the amino acid sequence of SEQ ID NO: 3.

9. The method of claim 6, wherein the synthetic peptide consists of the amino acid sequence of SEQ ID NO: 4.

10. The method of claim 6, wherein the synthetic peptide consists of the amino acid sequence of SEQ ID NO: 5.

11. The method of claim 6, further comprising administering to the subject an antiviral agent selected from the group consisting of berberine, glycyrrhizic acid, lactoferrin, kaempferol, melittin, minocycline, quinacrine, quercetin, reservatrol, ribavirin, suramin, ursolic acid, vapendavir, and xiyanping.

12. The method of claim 6, wherein the subject is a human.


SEQUENCE LISTING (11 sequences)

SEQ ID NO: 1 - protein, 21 aa, synthetic construct
  VARIANT 15: note = X is L or A
  VARIANT 16: note = X is S or A
  VARIANT 17: note = X is R or A
  VARIANT 20: note = X is S or A
PKKRRQRRRAYSRAXXXQLXS

SEQ ID NO: 2 - protein, 21 aa, synthetic construct
RKKRRQRRRAYSRALSRQLSS

SEQ ID NO: 3 - protein, 21 aa, synthetic construct
RKKRRQRRRAYSRALARQLSS

SEQ ID NO: 4 - protein, 21 aa, synthetic construct
RKKRRQRRRAYSRALSRQLAS

SEQ ID NO: 5 - protein, 21 aa, synthetic construct
RKKRRQRRRAYSRAAAAQLSS

SEQ ID NO: 6 - protein, 28 aa, synthetic construct
RKKRRQRRRYSREANQPKPSPKRESGEE

SEQ ID NO: 7 - RNA, 20 nt, synthetic construct
  source 1..20: mol_type = other RNA
  modified_base 4: mod_base = OTHER; note = utacil
  modified_base 12: mod_base = OTHER; note = uracil
  modified_base 14: mod_base = OTHER; note = uracil
  modified_base 15: mod_base = OTHER; note = uracil
gcatagccgcctcttcgacc

SEQ ID NO: 8 - DNA, 18 bp, synthetic construct
  source 1..18: mol_type = other DNA
cgcattgggcgaggtatc

SEQ ID NO: 9 - DNA, 20 bp, synthetic construct
  source 1..20: mol_type = other DNA
cggactgtaggcacctcgaa

SEQ ID NO: 10 - DNA, 22 bp, unidentified
  source 1..22: mol_type = other DNA
gtctcctctgacttcaacagcg

SEQ ID NO: 11 - DNA, 22 bp, synthetic construct
  source 1..22: mol_type = other DNA
accaccctgttgctgtagccaa